(19) United States
(12) Patent Application Publication
(10) Pub. No.: US 2012/0078310 A1
(43) Pub. Date: Mar. 29, 2012

(54) INFINITELY MODULAR PLATE CAGE DEVICE FOR FIXATION OF THE SPINE ABSTRACT

(76) Inventor: Avi Bernstein, Wilmette, IL (US)

(21) Appl. No.: 13/247,522

(22) Filed: Sep. 28, 2011

Related U.S. Application Data
(60) Provisional application No. 61/387,484, filed on Sep. 29, 2010.

(51) Int. Cl. A61B 17/88; A61B 17/70

(52) U.S. Cl. 606/279; 606/246

(57) ABSTRACT

The present invention discloses a device and method of application, combining a cervical plate system with a cervical graft (bone or synthetic) for the safe and efficient stabilization of the cervical spine. The application of a plate to the spine for fixation purposes is widely practiced. The present invention is designed to provide predictable, efficient, and safe fixation of the spine. The present invention is minimally invasive for the anatomical characteristics of the cervical bones or vertebrae. The present invention is designed primarily for use in the cervical spine, but can be applied to any level of application in the spinal column, including the thoracic and lumbo-sacral spine.

### CROSS-REFERENCE TO RELATED APPLICATIONS

This application is a continuation-in-part of U.S. provisional patent application No. 61/387,484 filed on Sep. 29, 2010 titled FIXATION DEVICE FOR THE CERVICAL SPINE.

### FIELD OF THE INVENTION

The current invention relates to the field of surgical implantation of spinal fixation devices and method, with applications for spinal fusion systems and all-level plate fixation. Fixation tools are of utmost necessity to a variety of pathologies, including elderly patients, those suffering from spinal disc degenerative diseases, the treatment of cervical disc herniations requiring fusion, the treatment of cervical instabilities resulting from trauma or degenerative disc diseases, or in the treatment of metastatic cancers invasive to the spine. The present invention and method is described, so one of ordinary skill in the art may understand the functionality and application of the present invention.

### BACKGROUND OF THE INVENTION

It is common practice for surgical medical professionals to attempt and correlate between the large variety of technological enhancements in the field of spinal fusion devices and procedures. It is largely understood by specialized medical audience such technologies do not always offer the desired ease of implementation. Many techniques of spinal fusion have improved the implantation of cervical fixation devices so patients may benefit from increased mobility and a certain degree of alleviation of their ailments. Spinal fusion is a surgical procedure used to “fuse” together the small sections of the vertebrae, so that motion between the vertebral sections is eliminated and the healing process proceeds as a single vertebral bone. Modular inter body spacers are known to have become yet another preferred device for surgical use while conducting spinal surgery. Modular spacers are designed primarily to accommodate the vascular anatomy or provide a method of containment of bone graft material and further facilitate spinal fusion procedures conducted by medical personnel (See, U.S. Pub. No. 2009/0306779).

The invented device represents an inter body fusion device. It is one element of an anterior spinal plating system. It is designed for anterior and lateral application to the spine (See, U.S. Pat. No. 7,306,605). Such characteristics of the present invention demonstrate a high level of applicability over the cervical spinal region, primarily, as well as ease of surgical implantation. The device can also be implanted and is applicable to the thoracic and lumbar spine. The present invention is an improvement over the prior art and inter-body fusion devices (See U.S. Pat. No. D 582,040, U.S. Pat. No. 7,641,701, U.S. Pat. No. 2010/0249937). The present invention promotes the safe spinal implantation and can be paired with a multiplicity of spinal fusion systems, including auto graft, allograft and synthetic (or metallic) cages. As a modular unit its design is easily customizable to accommodate a multiplicity of inter-body implant procedures and plate sizing. This allows the cervical fixation device to be paired with a variety of plate designs including static and dynamic plates hereby incorporated and attached, as well as fixed or variable angled screws. Because of the plate-graft relationship, a predictable and reproducible alignment of the plate with the spine is achieved by the present invention. A variety of plate lengths allows for screw fixation through the plate at either corner of the anterior cortex of the vertebral body. The pivoting plate and graft holder join and allow the plate to nestle itself in an anatomically acceptable position matching a variety of anatomical variations, and the assembled implant, once impacted, requires no pre-drilling. The plate can be used with either static or multi-axial screws. Additionally, the plate can be designed to mate with a variety of screw plate-looking mechanisms.

### SUMMARY OF THE INVENTION

A method and tool are provided for facilitating the sizing, orientation and implantation of spinal devices that are minimally invasive of the anatomic characteristics of spinal bones such as the vertebrae. Said method and tool are designed to aid in the placement of implantable cervical plates and cervical grafts at virtually all spinal levels.

In an alternate embodiment, the instant invention is designed so the surgeon determines the specific graft limitations of the patient during surgery. Further, micro adjustments are performed by the surgeon based on a previous routine discectomy or corpectomy procedure to determine ideal graft positioning.

Further, because specific features of a patient's spinal anatomy can vary significantly from patient to patient, an implantable spinal device will be configured to be patient-specific in order to accommodate the specific features of the patient's spinal anatomy.

### BRIEF DESCRIPTION OF THE DRAWINGS

The foregoing and other advantages of the invention and related methods will be appreciated more fully through consideration of the accompanying drawings wherein:

FIG. 1 depicts a side view of a screw through a plate which attaches to the vertebrae. The graft holder is shown pivoting at the graft holder plate junction.

FIG. 2 depicts a frontal view of a plate with central holes where the graft holder can snap to the plate.

FIG. 3 depicts a simple attachment of a graft to a plate.

FIG. 4 depicts a side view of the pivot point at the base of a graft as opposed to FIG. 1 where the pivot point is at the plate graft holder articulation.

FIG. 5 depicts an angle of the plate which uses a ball socket and hinge to become adjustable.

FIG. 6 depicts a top view of a graft holder demonstrating one of a number of mechanisms attaching the graft to the graft holder while necessary adjustments can be performed to accommodate different sizes.

FIG. 7 depicts instrumentation, which would determine the graft height and depth with adjustable characteristics.

FIG. 8 depicts top and side views of the graft being attached to the plate.

FIG. 9 depicts a side view of a plate attached to the vertebrae while a non-pivoting graft holder is inserted into the graft material.

### DETAILED DESCRIPTION

As observed through a method and tool for facilitating the sizing, orientation and implantation of spinal devices is provided. Said invention and methods are minimally invasive and non-invasive of the anatomic characteristics of bones such as the vertebrae.

FIGS. 1, 2, 9 show the placement of implantable cervical plates and cervical grafts and further application to the spine (including the cervical, thoracic and the lumbar spine) wherein a single implant can be used at each level. This allows the invention to become independent of the adjacent level so that multiple implants can be used in one single operation, including an alternative comprising a long plate with multiple graft holder articulations with an element of vertical adjustment to allow variable distance between the grafts which accommodates a multi-level application.

Referring to FIG. 6, it can be observed that the present invention may act as a modular unit which can be mated to a multiplicity of inter body devices including auto graft, allograft and synthetic and metal cages which are customizable depending on individual needs. In terms of precise inter body implant placement and plate sizing the device may be paired to a variety of plate philosophies including static and dynamic plates. The plate-graft relationship allows a predictable, reproducible alignment of the plate with the spine to be achieved, and the significant variety of plate lengths allows for screw fixation through the plate at both the corner and the anterior cortex of the vertebral body.

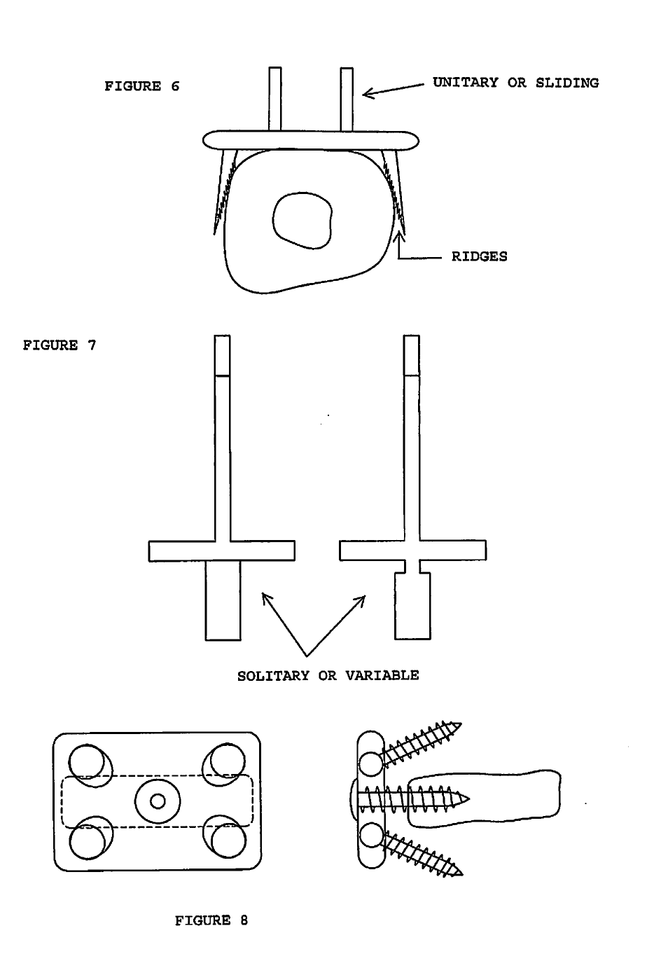

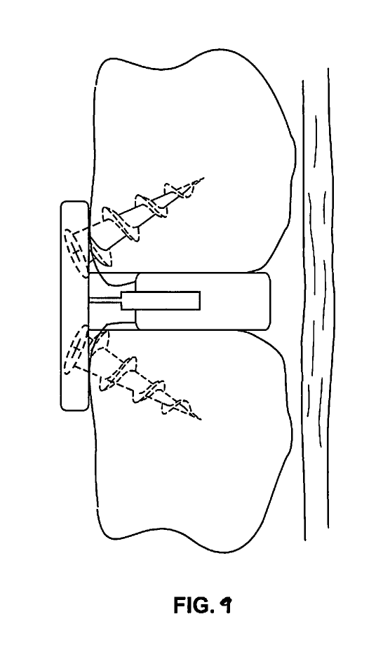

FIGS. 1, 4 depict the pivoting plate and the graft holder articulation which allow the plate to nestle itself in an anatomically acceptable position matching a variety of anatomical variations, wherein the assembled implant, once impacted, requires no pre-drilling, the plate designed to mate with a variety of screw plate locking mechanisms. FIG. 9 further depicts a non-pivoting side view of the graft holder, with screws attaching the plate to the vertebrae and the graft holder attached to the graft material. The spinal cord is also depicted so that an exemplary view of the spinal fusion procedure is depicted.

Referring to FIG. 5, a single or dual receptacle is observed, which allows for the attachment of the graft holder, wherein the articulation between the plate and the graft in a first facsimile can be static and defined by a right angle relationship on another fixed angle. Alternatively, the plate and the graft holder articulation allow cephalo-cauded (head to toe) angulation so that an insertion of the plate adapts to the spinal anatomy accommodating a small degree of off-set.

Furthermore, there is no motion between the stalks of the graft holder and the plate, there is motion through the articulation between the graft holder and the stalks, and there is also motion between the base plate of the graft holder attached to the graft and the stalks. The degree of rotation through this articulation can be mechanically fixed by stabilizing the graft stalks with a small cross bar, the lengths of the stalks coming in a variety and multiplicity of sizes to match the variety of anatomical requirements. Another characteristic permits for the stalks to become telescopic to allow for a variable graft depth, pre-determined implantation (See, FIG. 7).

Through FIG. 6, it can be observed how the end of the graft holder, which mates with the bone graft, simply holds onto the graft through a ratcheted compression mechanism, wherein the length of the side arms which hold the graft can be defined as either being smooth or with ridges and stabilizing the graft through compression of the ratcheted compression mechanism. In yet another embodiment, the graft holder can be glued to the bone graft using a variety of biological glues, or the graft holder can be screwed to the graft. The graft holder can be made from a variety of materials, either metallic or synthetic, wherein the flexibility of the material allows grasping of the bone graft. Said bone graft can be defined to match the surgeon's philosophy whether consisting of auto graft bone such as the tri-cortical iliac crest bone, graft bone, pre-fabricated allograft or synthetic cage, which can consist of a variety of materials including metals, PEEK™, ceramic, carbon fiber or another material.

The surgical procedure comprises a routine discectomy or corpectomy while intervertebral pre-fabricated spacers which allow variable depth are used as trials. Said spacers will determine ideal graft positioning, wherein the total depth from the edge of the vertebral body is measured and a bone graft and graft holder are selected to match the identical depth using a construction tray. The above mentioned process will allow attachment of the graft holder to the graft. Further, the plate length is selected based on trials and surgeon's preference, and then become attached to the graft holder; the implant is completely assembled and positioned, whilst the screw sites are prepared. Said screws are placed into the vertebral bodies and a locking mechanism stabilizes the screws to the plate.

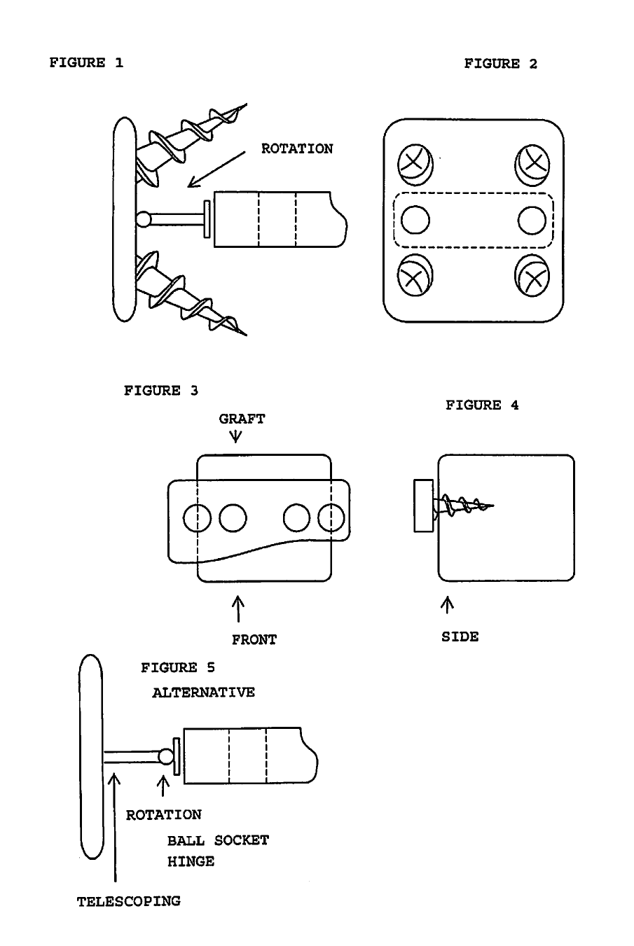

Referring to FIG. 2, the plate may be observed with fixation holes at the corners to apply screws to the cervical spine, where the screws are placed through the corners of the anterior position of the vertebral body to fix the plates to the spine. The plate is attached to the graft by one of a number of mechanisms, where one mechanism would be to have a plate graft assembly, where drilling and screwing of the graft to the plate is performed. A second method would be to have a bar across the middle of the cervical plate that allows, through a turnbuckle, variable graft depth placement, or third method, would be a ridge projection from the plate that would grasp the graft. The pre-assembled graft-plate relationship allows an angular adjustment between the graft and the plate. Variable depth and the attachment of various grafts to the plate in a modular fashion are possible.

A novel relationship is distinguished between an inter body device (referred to as a bone graft), an articulating member (referred to as graft holder), and an anterior spinal plate. The plate can be considered a typical anterior spinal plate, which can be static or dynamic. It is distinguished by the center of the plate facing the spine having a single or dual receptacle that allows attachment of the graft holder. Once the graft dimensions are determined from the surgical patient during surgery, a specific graft is selected from available grafts and attached to the graft holder. The graft and graft holder are then attached to the plate. The plate graft is impacted into position on the front of the cervical spine; micro adjustments in graft depth can be performed at that time and evaluated at the time of the surgery.

The inventive device can be utilized in discectomy and corpectomy applications. A single implant can be used at each spinal level independent of the adjacent level so that multiple implants can be used in a single operation. As an alternative, one can visualize a long plate with multiple graft holder articulations with an element of vertical adjustment to allow variable distance between the grafts to accommodate a multi-level application. As another alternative, a long plate can be mated to a single graft holder where only a single level accepts the graft holder, and the remainder of the plate acts as a buttress in a multi-level application.

In summary, the proposed advantages of the current invention are:
1) The system allows safe, reproducible and predictable inter body graft positioning and plate alignment using a specific assembled implant;
2) The system is modular; customizable graft depth with ideal graft placement is achieved in a safe and predictable manner;
3) Variable plate graft angular relationships allows for adaptation of the implant to the patient's anatomy;
4) The plate can be defined as static or dynamic;
5) The plate can be fixed for either anterior corner or anterior vertebral body fixation;
6) Ideal plate positioning and alignment is achieved in a reproducible fashion;
7) Drilling is accomplished after implantation obviating the need for pre-drilling; and
8) The system allows a multiplicity of bone graft choices.

What is claimed is:

1. A cervical fixation device for the spine comprising: a combination interbody fusion device (graft holder and graft) and an anterior spinal plating system designed for anterior application to the spine.

2. A method for the implantation of spinal fixation devices facilitating the sizing, orientation and implantation of spinal devices that is minimally invasive of the anatomical characteristics of bones such as the vertebrae.

3. The cervical fixation device of claim 1 wherein lateral application to the spine can be achieved.

4. The cervical fixation device of claim 1 wherein the system can be a modular unit and become mated to a multiplicity of interbody devices including auto graft, allograft and synthetic or metal cages.

5. The cervical fixation device of claim 1 wherein the plate component can be defined as static or dynamic, at either corner or anterior cortex of vertebral body.

6. The cervical fixation device of claim 1 wherein the pivoting plate and graft holder articulation allows the plate to become situated in an anatomically correct position without requiring pre-drilling.

7. The cervical fixation device of claim 1 wherein the plate component is designed to mate with a variety of screw-plate locking mechanisms while the plate has a single or dual receptacle allowing attachment of the graft holder.

8. The cervical fixation device of claim 1 wherein the plate articulation between the plate and the graft can be static and defined by either a right angle or cephalo-cauded (head to toe) angulation.

9. The cervical fixation device of claim 1 wherein the plate articulation can be rotated and mechanically fixed by stabilizing the graft stalks while said stalks have telescopic characteristics which allows for variable graft depth.

10. The method of claim 2 wherein the surgeon may attach the graft holder to the graft while the graft may be stabilized through compression of the ratcheted compression mechanism and a variety of materials may be chosen to comprise the graft. The graft holder can be constructed of a variety of materials, including metal or synthetic material with varying degrees of flexibility, as an alternative mechanism to attach to a graft.

11. The method of claim 2 wherein a routine discectomy or corpectomy is performed having intervertebral pre-fabricated spacers providing for variable depth, allowing for the determination of a total depth, wherein said spacers are used as trials so that an ideal graft positioning is determined the total depth from the edge of the vertebral body is measured, and a graft holder and a bone graft are selected to match the identical total depth using a construction tray providing for attachment of the graft holder to the graft.

12. The method of claim 2 wherein the plate length is selected based on trials and the surgeon's preference wherein the graft is attached to the graft holder; the implant becomes completely assembled and set into position and the screws are placed into the vertebral bodies while a locking mechanism stabilizes the screws to the plate.
